(19) United States
(12) Patent Application Publication
(10) Pub. No.: US 2012/0058516 A1
(43) Pub. Date: Mar. 8, 2012

(54) SEALING MULTIWELL PLATES

(75) Inventor: Peter Collins, Surrey (GB)

(73) Assignee: 4Titude Limited, Ockley Surrey (GB)

(21) Appl. No.: 13/203,656

(22) PCT Filed: Mar. 2, 2010
(86) PCT No.: PCT/EP2010/052629

§ 371 Date: Nov. 17, 2011

(30) Foreign Application Priority Data
Mar. 3, 2009 (GB) 0903623.7

(51) Int. Cl. C12P 19/34; B32B 38/10; B01L 99/00; B32B 37/06

(52) U.S. Cl. 435/91.2; 156/292; 156/247; 422/569

(57) ABSTRACT

The present invention relates to a heat sealable member comprising a sheet of heat sealable material to a multiwell plate, wherein the sheet is mounted in a frame, and wherein said frame is sized to fit over a multiwell plate so that the sheet is contacted with at least one of the rims of the top of the plate and the individual wells; as well as to the use of the heat sealable member with automatic plate handling apparatus, especially in PCR processes. The present invention further relates to a method for sealing multiwell plates.

### TECHNICAL FIELD OF THE PRESENT INVENTION

This invention relates to the sealing, permanently or on a temporary basis, of wells in a multiwell plate.

### BACKGROUND OF THE PRESENT INVENTION

Multiwell plates, namely a plate provided with a plurality of individual wells in its upper surface, are widely employed for the storage of samples and reagents, and for the preparation, treatment and analysis of samples, in the chemical and biological science fields. Often it is necessary or desirable to cover some or all of the wells in the plate. A variety of means have been used to achieve this, including caps, lids, mats, adhesive seals and heat seals. A typical closure means and method of closure for containment devices such as reagent containment devices which rely upon a heat seal are disclosed in WO 94/12405 of Techne (Cambridge) Ltd.

Over the years, a variety of different heat seals have been developed for different applications. These have included materials made up of multiple layers, including a heat seal layer to give a seal that is permanent or may be temporary, being peelable, to the (usually) different polymer from which the multiwell plate is made, and a barrier layer such as a foil to reduce or eliminate permeability through the seal to gases or vapours. A support layer with a higher thermal melt temperature compared to that of the seal layer may be included to give the seal integrity. In some instances seals are clear to enable optical inspection of the contents of a sealed well.

In a manual heat sealer, a sheet of heat sealable material cut to size is applied to the top of the multiwell plate and a heated block brought down into contact with the top surface of the film to fuse its lower surface to the plate and preferably about the rim of each individual well.

Heat sealable material can readily be supplied in a roll form and will not stick to anything else until heated. This has led to the development of automatic heat sealers in which a length of heat sealable material is withdrawn from a roll of such material, cut to size, applied to the top of a multiwell plate, and heat sealed, all automatically.

Although such automatic plate sealers are widely employed in laboratories they suffer from a number of disadvantages. Firstly, because they employ the several actions of withdrawing material, cutting to size, applying and heat sealing, these machines tend to be both complex and costly. Secondly, they are not ideal for some uses such as diagnostics or forensics, where quality control and track-ability assume greater importance. Most heat seal components are made in facilities that are not necessarily of “clean room” standard, and rolls of heat sealable material cannot easily be sterilized. Accordingly, with conventional equipment, it is difficult to get a high degree of quality assurance with automatic heat sealing. The alternative would be to use individual sheets of heat sealable material, but these are difficult to handle in an automated way, while manual application raises the possibility of contamination unless performed very carefully.

While some materials allow for a heat seal once applied to be removed by peeling, a second application of heat to soften the previous seal, followed by peeling of the material is not easy to achieve, and the seal is usually incapable of re-use.

As will become clear from the detailed description of preferred embodiments below, we have sought to address the several disadvantages noted above.

### DESCRIPTION OF THE PRESENT INVENTION

In accordance with a first aspect of this disclosure, there is provided a heat sealable member comprising a sheet of material heat sealable to a multiwell plate, the sheet being mounted in a frame, the frame being sized to fit over the multiwell plate so that, when so fitted, the sheet makes contact with at least one of the top of the plate and rims of individual wells therein.

In a second and alternative aspect of this disclosure, a method is provided for sealing a multiwell plate or one or more wells therein, the method comprising the steps of placing a frame in which a sheet of heat sealable material is mounted over the top of a multiwell plate and applying heat to the sheet or to at least a selected portion thereof to seal the plate as a whole or one or more wells therein.

The sheet may be fixedly mounted in the frame so that the frame remains after heat sealing. Alternatively, the frame may be detachable from the sheet, for example as the result of softening of the sheet during the heat sealing step, so that the frame may be removed leaving the sheet sealed in position. Where the frame remains, a subsequent softening of the sealed material may allow the sheet, still mounted in its frame, to be removed without needing to touch the sheet itself, allowing access to contents of one or more wells. Thereafter, the sheet, still mounted in its frame, may then be reapplied by heat sealing.

A variety of ways of mounting the sheet in a frame are contemplated. Thus, the sheet may be welded or glued to the frame; the sheet may be held on integral pins of the frame; the frame may be in two parts that fit together sandwiching portions of the sheet between them; the sheet may be releasably clipped to the frame. For reasons of economy, we prefer mounting arrangements that allow frames to be re-used by mounting a fresh sheet in a similar manner.

Since the sheet is mounted within its frame during any handling steps, there is no need for any handling of the sheet itself either manually or automatically, except by a heat seal platen, all handling being by way of the frame, thereby avoiding contamination.

The frames are preferably stackable without the sheet of one frame making contact with any part of an adjacent frame in the stack.

The frame is preferably formed from a relatively rigid polymer such as a filled polycarbonate or polystyrene. It may have a skirt beneath the sheet and an upstanding wall above the sheet, the skirt terminating in a lower rim adapted to sit over the upper edge of the upstanding wall of an underlying frame in a stack.

Other materials from which the frame may be formed in alternative embodiments include wire and cardboard.

### DETAILED DESCRIPTION OF THE PRESENT INVENTION

The multiwell plate 1, shown in FIGS. 1 to 4, comprises a plate 2 with an integral skirt 3, the plate mounting an array of individual thin walled wells 4. Lower edge 5 of skirt 3 is formed with an outwardly displaced rim 6, enabling the plates to be stacked one upon the other with the displaced rim fitting over the edge between plate 2 and skirt 3 of an underlying multiwell plate in the stack. The individual wells 4 are mounted in openings in the plate 2 with rims 7 extending above the surface of the plate 2. The plate has a chamfered corner region 8 and cut-outs 9 for indexing purposes. The design of multiwell plates is subject to a number of Standards, such as the SBS Standard, in order for multiwell plates from different manufacturers to be compatible with automatic plate handling apparatus, for example for performing the PCR (polymerase chain reaction) process; but persons skilled in this art will know that a number of variations are commonly encountered, including semi-skirted and non-skirted plates, plates in which the wells are integrally formed with the plate as well as arrangements in which the wells are formed from a material that differs from that of the plate and are fixed in position in openings therein. The number, size and disposition of the wells may differ, although the 8 by 12 array illustrated is common. In order that the wells and their contents may be subject to repeated thermal cycling in a PCR protocol, the wells at least are formed of a plastics material that generally resists distortion or melting at the temperatures likely to be encountered.

As explained hereinabove, various forms of apparatus for manually or automatically heat sealing multiwell plates are available commercially. All employ either individual sheets of heat seal material or a roll of heat seal material from which a length is drawn, cut to size, applied to the top of a plate and heat-sealed thereto. Heat sealing is performed by a heated platen applied manually, automatically or semi-automatically.

Turning now to FIGS. 5 to 9, there is shown a heat sealable member 10 comprising a sheet 11 of heat sealable material, otherwise of conventional form for sealing to multiwell plates, except that it is mounted in a frame 12. Frame 12 is designed to fit over the top of a multiwell plate 1, for which purpose it has a chamfered corner 13 corresponding to that of skirt 3 of the multiwell plate. Frame 12 has a skirt portion 14 that extends beneath the sheet 11 and, in this embodiment, but not always necessary, also has an upstanding wall 15 that extends above the sheet 11, the skirt portion terminating in a lower rim 16, best shown in the sectional view of FIG. 9) displaced outwardly from the remainder of skirt portion 14. The edge of wall portion 15 is tapered slightly. As a result, the illustrated members 10 can be stacked one above the other with the displaced rim 16 of one frame 12 located on the edge of the upstanding wall 15 of an underlying frame 12 in a stack of the members 10, without the sheet 11 of one member 10 making contact with any part of another member 10. Individual members 10 may thus be handled either manually or automatically by robotic equipment by their frame 12 without compromising the sterility, integrity and accountability of their respective sheets 11.

In this embodiment, frame 12 is integral with its sheet 11, for example being formed of the same material as the support layer or of the same material as the sealing layer of a multilayer sheet 11. The frame is suitably formed from a relatively hard plastics material such as a filled polycarbonate, polypropylene or polystyrene, but may be formed from metal wire or from cardboard.

The skilled artisan will recognize that a “multilayer sheet” comprises at least two layers, e.g. a support layer and a heat sealable layer.

FIG. 10 illustrates an alternative embodiment in which frame 12 is formed from two portions 17 and 18 of cardboard between which sheet 11 is sandwiched.

In use, in order to seal a multiwell plate 1 or one or more of its individual wells, a member 10 handled only by its frame 12 is placed over the multiwell plate 1 so that the heat sealable face of its sheet 11 makes contact with the top of the multiwell plate. Heat is suitably applied by a heated platen of a manual or automatic plate sealer being brought into contact with the reverse side of sheet 11 to press it against confronting surfaces of the multiwell plate with application of heat so that the heat sealable layer of sheet 11 is softened and fuses to surfaces of the mu ltiwell plate, preferably to the upstanding rims 7 of the individual wells. Heat may be selectively applied by the platen to seal the sheet only to some of the wells. In alternative arrangements, depending on the formulation of the heat sealable layer of sheet 11, the sheet may be sealed by application of hot air to the reverse of the sheet. By directing hot air at portions of the sheet above to only some of the wells, only those wells may be sealed.

Prior to such sealing step, the member 10 acts effectively as a lid for the multiwell plate, avoiding inadvertent contamination.

Application of heat to the sheet 11 may cause such softening of the sheet that it detaches from its supporting frame. Alternatively, the heat sealing platen may be provided with sharp edge regions that cut the sheet from its supporting frame. In other arrangements, the frame remains in position, sitting neatly over skirt 3 of the multiwell plate with cut-outs 19 positioned so that at least some of the cut-outs 9 of the multiwell plate remain accessible.

Preferably, the sheets 11, having been sealed to a multiwell plate, are capable of being unsealed by application of heat to soften the heat sealable layer of the sheet. Provided that the sheet 11 is still supported by its frame 12, we have found that it retains sufficient integrity for the member 10 as a whole to be removed from the multiwell plate, for access to one or more of the individual wells, after which the sheet 11 may be resealed to the same plate. This procedure avoids the handling difficulties, risk of contamination that occurs if a conventional sheet is softened and peeled away for access to one or more wells. Whereas such prior sheets could not in general be reused, we have found that otherwise identical sheets 11 mounted in frames 12 in accordance with the present teachings are readily capable of resealing use.

Although sealing is described hereinabove in connection with a multiwell plate 1 having a full skirt, it will readily be understood that embodiments of heat sealable member 10 with a suitable frame supporting a sheet 11 may be employed with other forms of multiwell plate having either a partial skirt or no skirt at all.

Turning now to FIGS. 11-15, which correspond to each of FIGS. 5-9 respectively, an alternative embodiment is illustrated in which the sheet 11 is welded or glued to the frame 12, which, in this embodiment, includes a circumextending ledge 20 around its inner side to the surface of which the sheet 11 is glued or welded. In this embodiment the materials of the ledge and sheet preferably differ so that, upon the application of heat, the connection between sheet and ledge, whether provided by welding or gluing is loosened to the extent that the frame can be readily separated from its sheet, which remains, now attached to the multiwell plate. Alternatively, a heat sealable member with the geometry shown in FIGS. 11-15 may have a frame that remains in position after sealing the sheet to the multiwell plate.

FIGS. 16-20, which correspond to each of FIGS. 5-9 respectively, show another embodiment of frame, in which the ledge 20 is furnished with a plurality of sharp spikes or pins 21. In this embodiment, although not necessarily so, spikes or pins 21 are formed integrally with ledge 20. An edge portion of a sheet 11 is impaled on the pins or spikes 21 to attach the sheet to the frame. The attachment is only temporary so that, following application of heat to the sheet to attach the sheet to at least one of the top of a multiwell plate and rims of wells therein, the frame may readily be detached from its sheet, which is left in place attached to the multiwell plate, by a sharp downward push on the frame 12.

In the arrangement of FIGS. 21-25, the frame is formed in two parts, namely a first frame portion 12, generally similar to the frame of FIGS. 16-20, except that the sharp pins or spikes 21 are replaced by studs 22, and an apertured second frame part 23 with through openings 24 that correspond to the positions of the studs 22, so that the second frame part may clip over studs 22 to complete the frame. The sheet 11 has apertures 24 corresponding to studs 22 and is fitted over studs 22 and sandwiched between the two frame portions. In use of this embodiment, once heat has been applied to the sheet to attach it to at least one of the top of a multiwell plate and rims of wells therein, the two frame portions may be readily detached by pushing the main frame portion 12 sharply downward, the sheet being left in place on the multiwell plate.

FIGS. 26-30 show yet another embodiment of frame and sheet combination. Sheet 11 has apertures 24 as in the arrangement of FIGS. 21-25, and main frame portion 12 has studs 22 as in the arrangement of FIGS. 21-25. However, in place of a second frame part 23, this embodiment employs discrete clips 26 for each stud, the clips holding the sheet in place, but being detachable from their respective studs 22 by a sharp downward push on the main frame portion similar to that employed with the embodiment of FIGS. 21-25.

The sharp downward push on the frame called for in use of embodiments described above may be provided automatically by the plate sealing apparatus, and may follow immediately after and in a continuation of the movement of the machinery to apply heat and pressure to the sheet.

### FIGURE LEGENDS

Reference will now be made, by way of example only, to the accompanying drawings, in which:

FIG. 1 is a plan view of an exemplary multiwell plate;

FIG. 2 is a side elevational view of the plate shown in FIG. 1 as seen from the left;

FIG. 3 is a cross-sectional view taken along the line in FIG. 1;

FIG. 4 is an enlarged scrap sectional view showing a single well;

FIG. 5 is an over-all perspective view of an embodiment of heat sealable member;

FIG. 6 is a similar over-all perspective view as seen from below;

FIG. 7 is a top plan view of the member of FIGS. 5 and 6;

FIG. 8 is a sectional view taken along the line VIII-VIII in FIG. 7;

FIG. 9 is an enlarged view of the portion shown ringed in FIG. 8;

FIG. 10 shows the corresponding portion of an alternative embodiment;

FIGS. 11-15 correspond respectively to each of FIGS. 5-9 for a further combination of sheet and frame;

FIGS. 16-20 correspond respectively to each of FIGS. 5-9 for a another combination of sheet and frame; and

FIGS. 21-25 correspond respectively to each of FIGS. 5-9 for a yet another combination of sheet and frame.

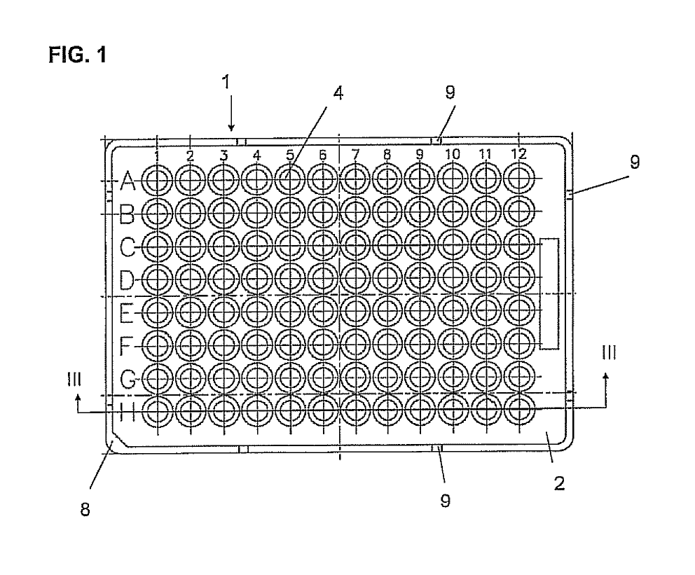

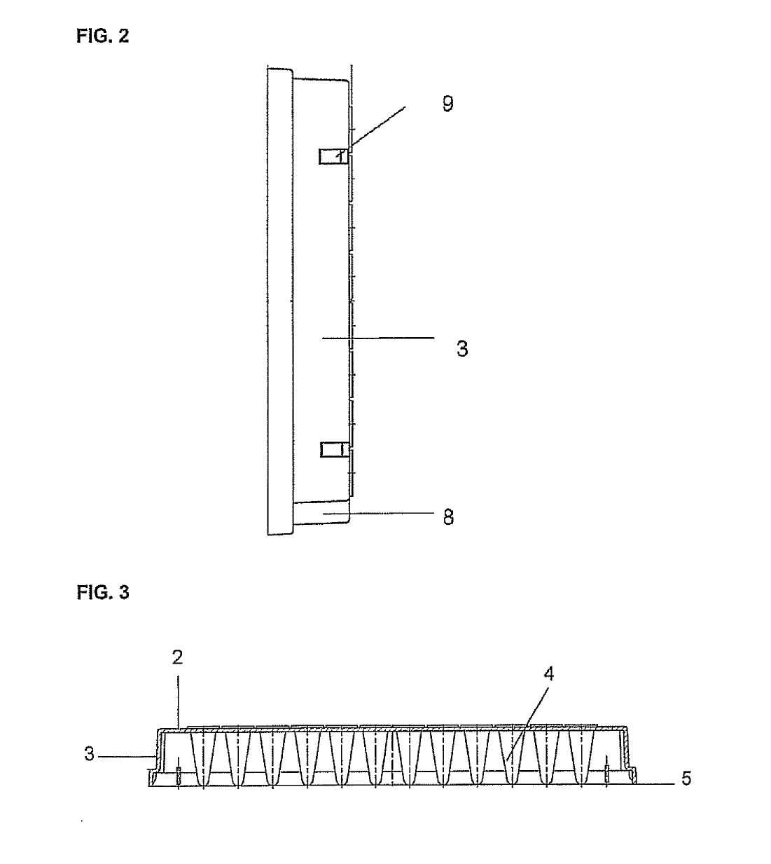

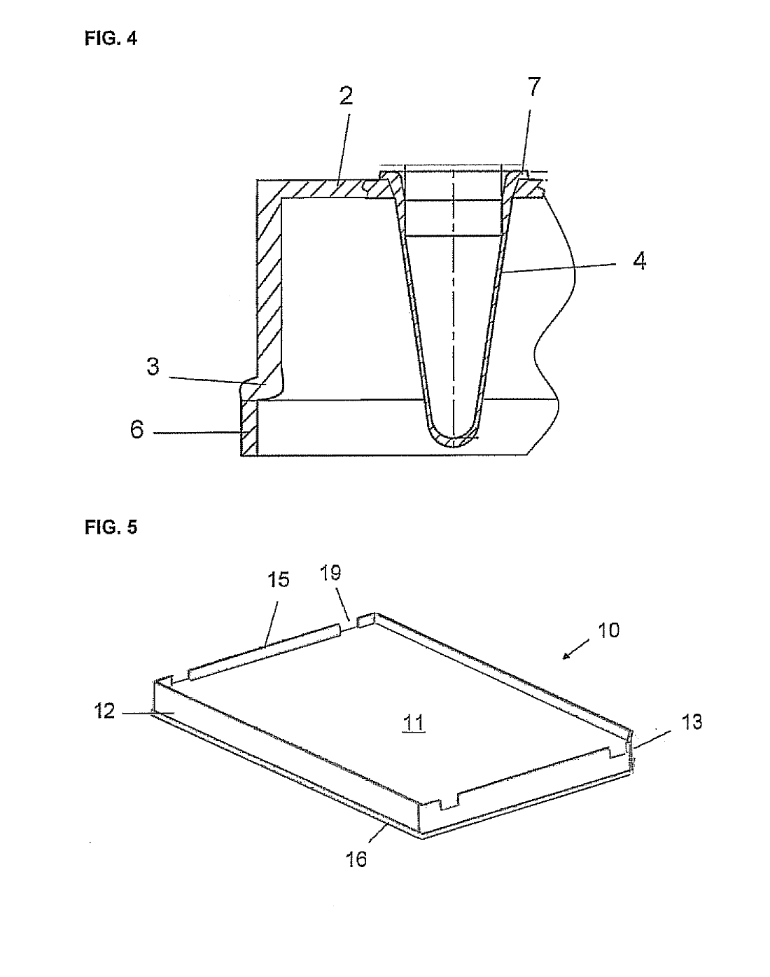

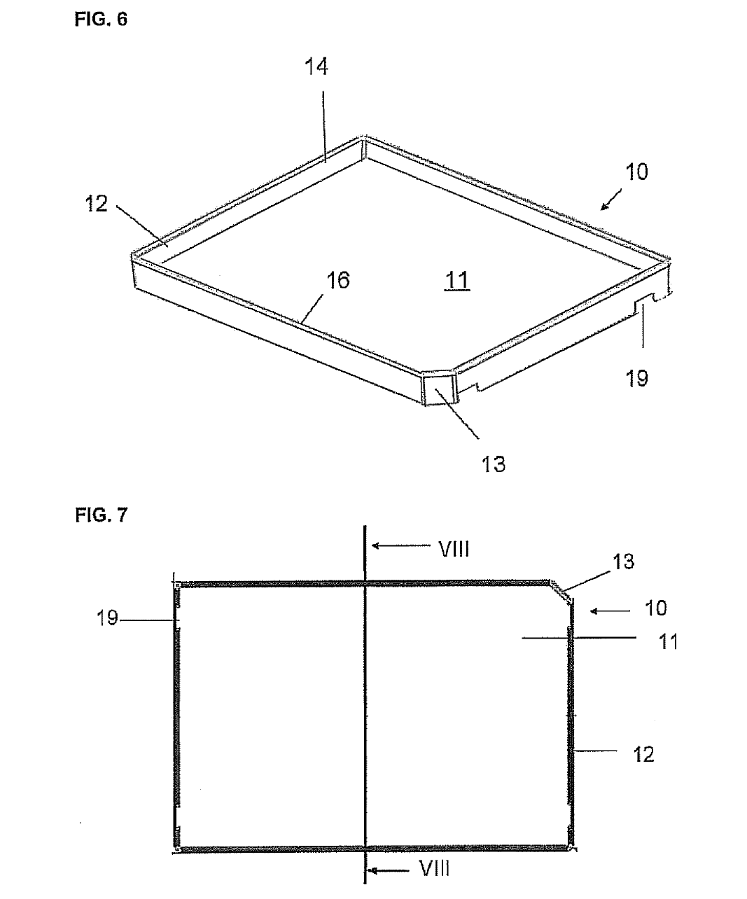

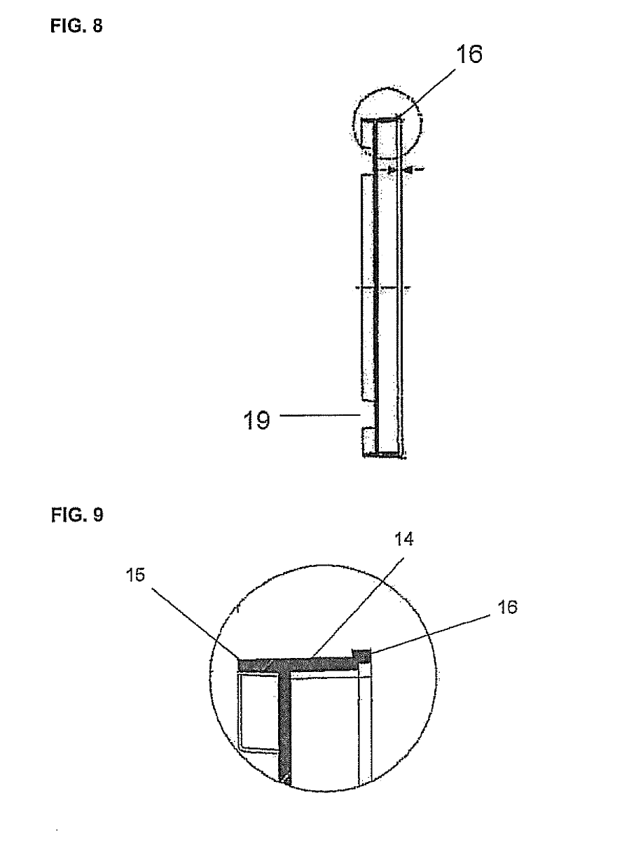

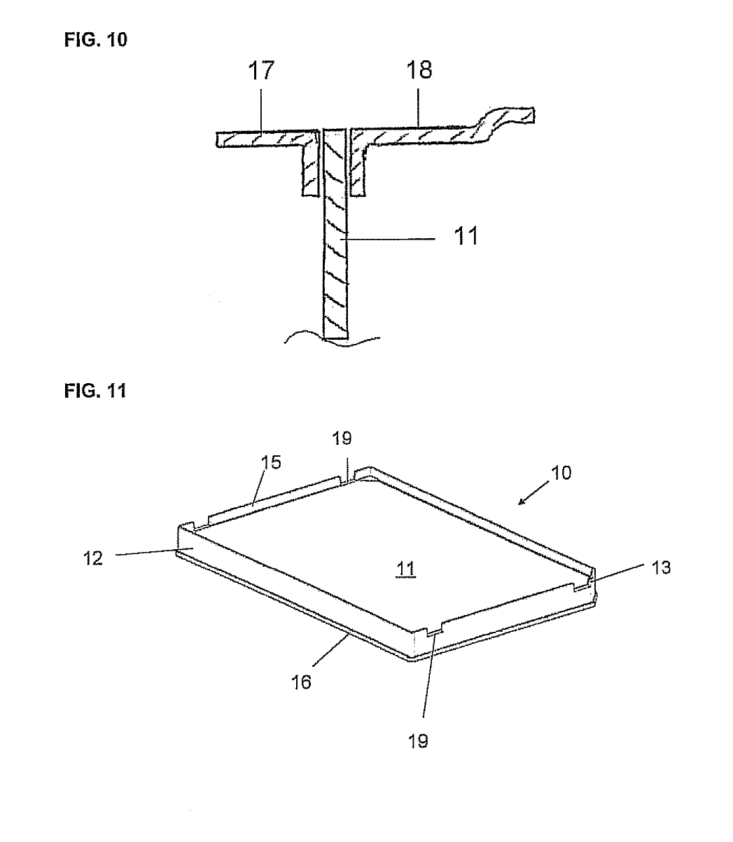

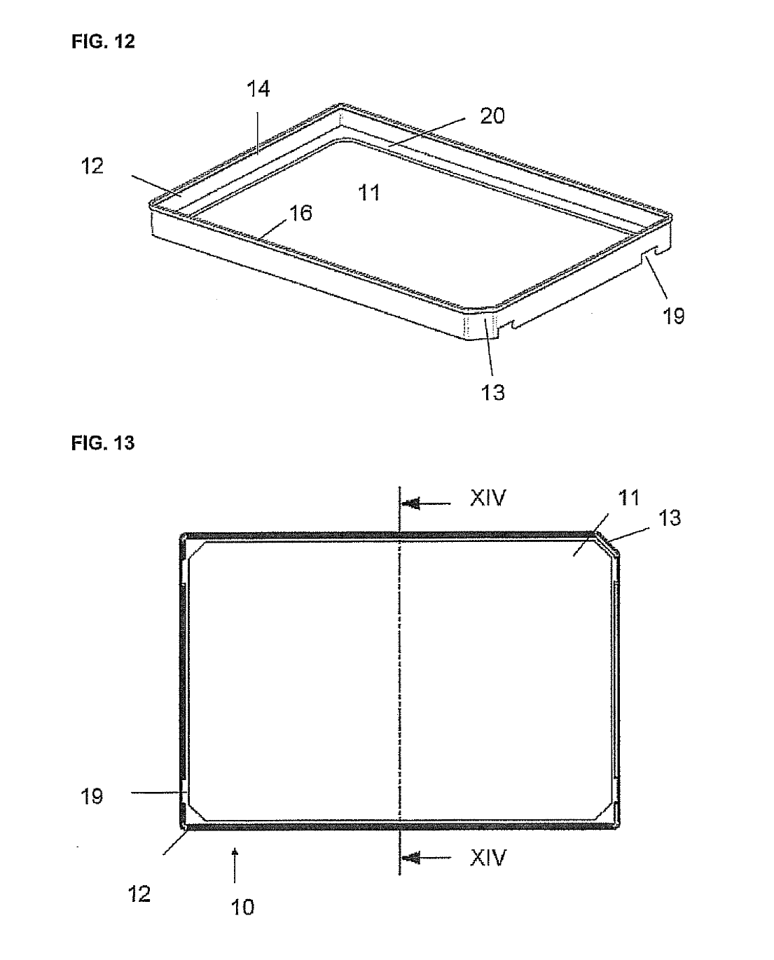

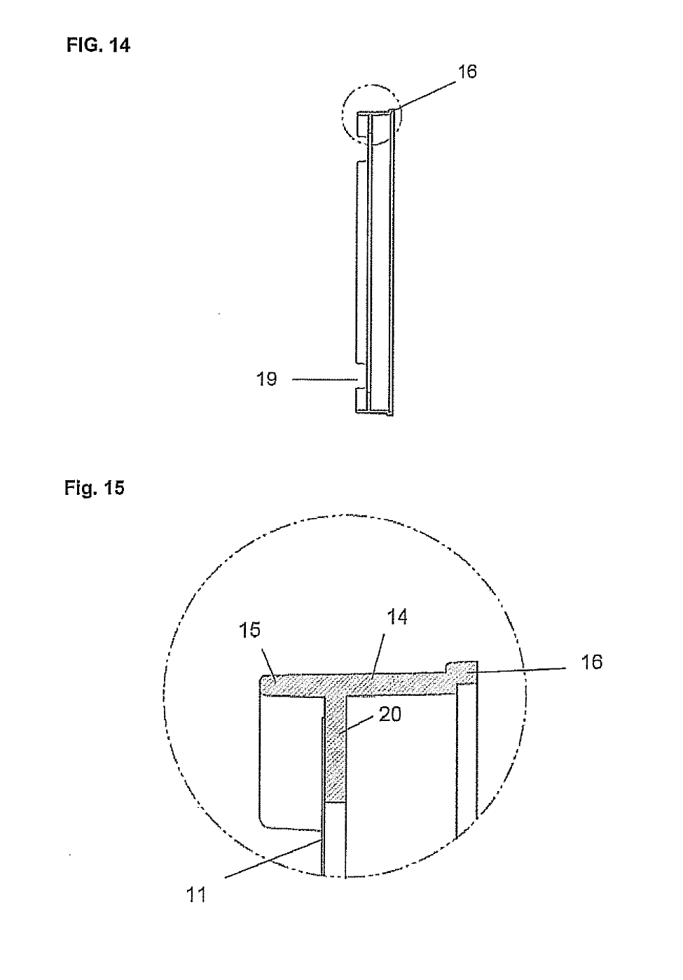

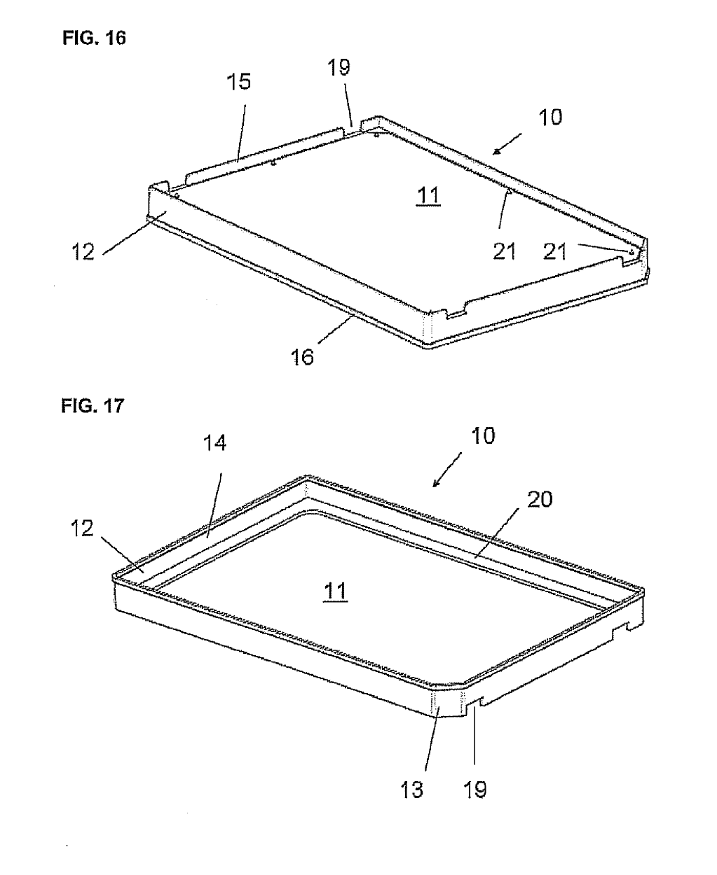

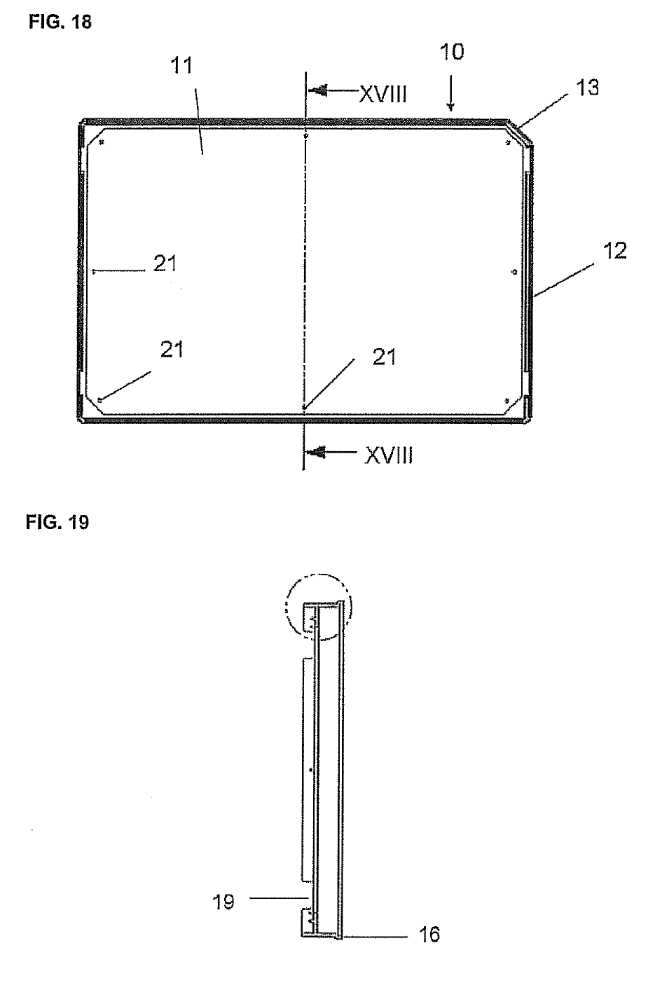

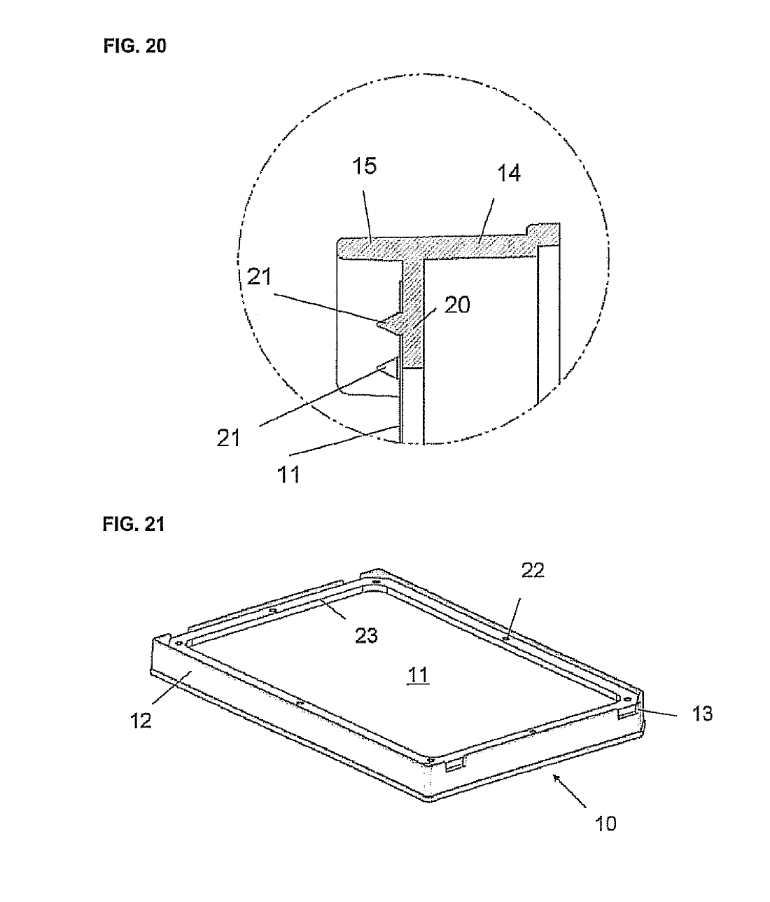

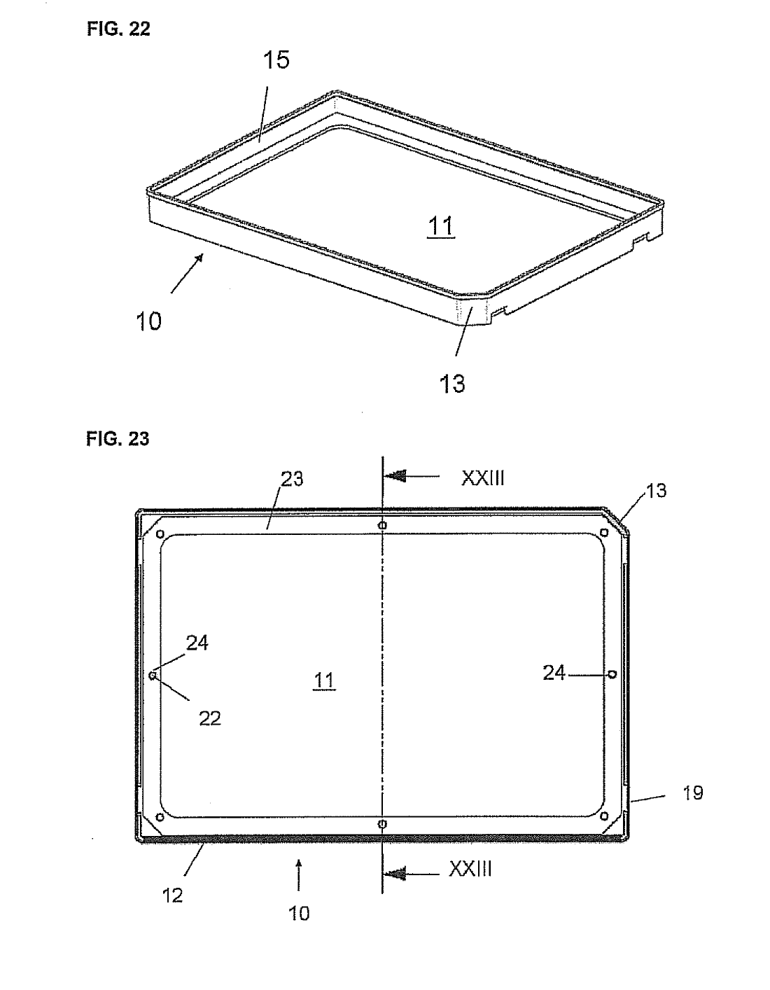

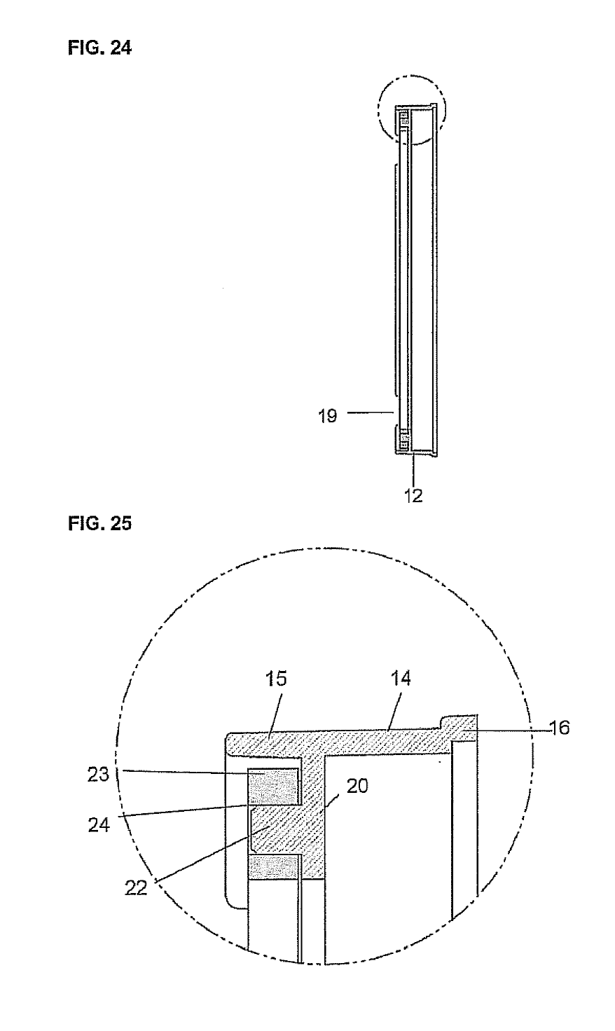

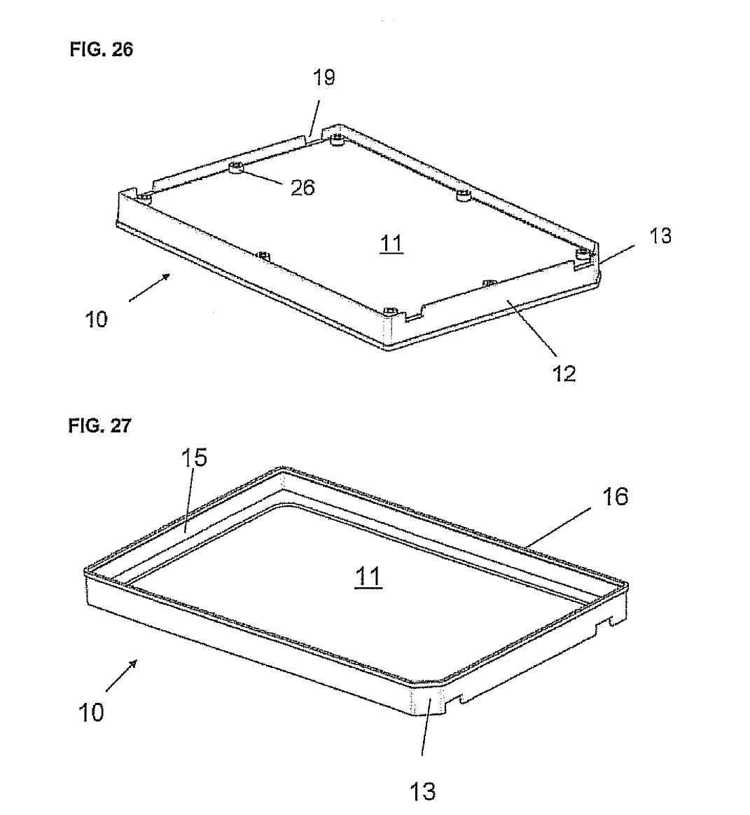

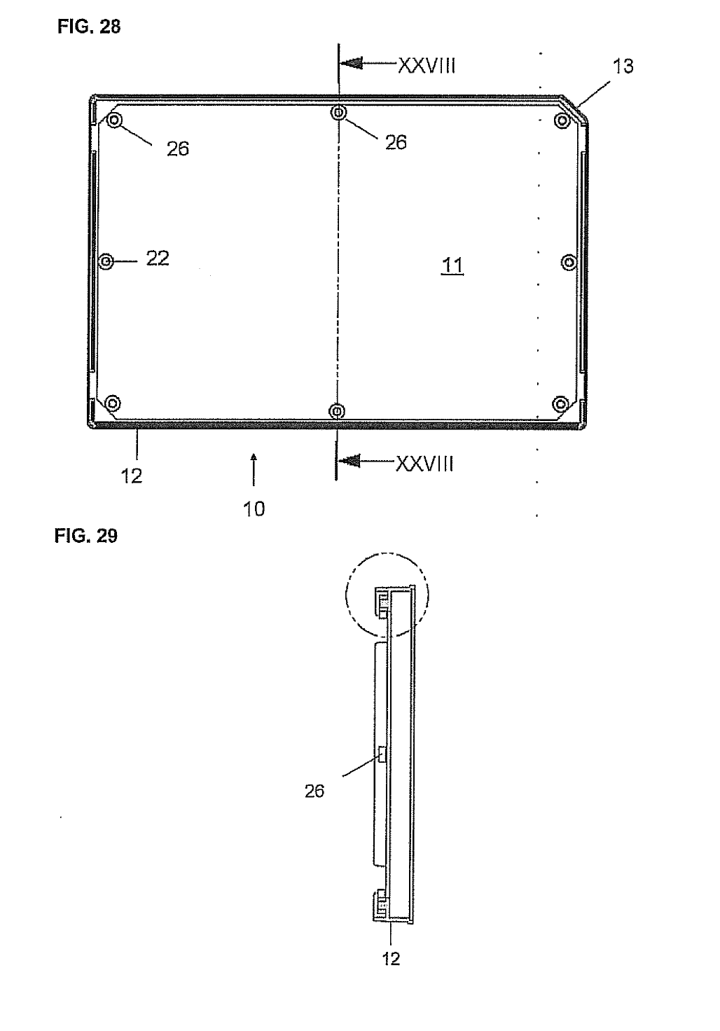

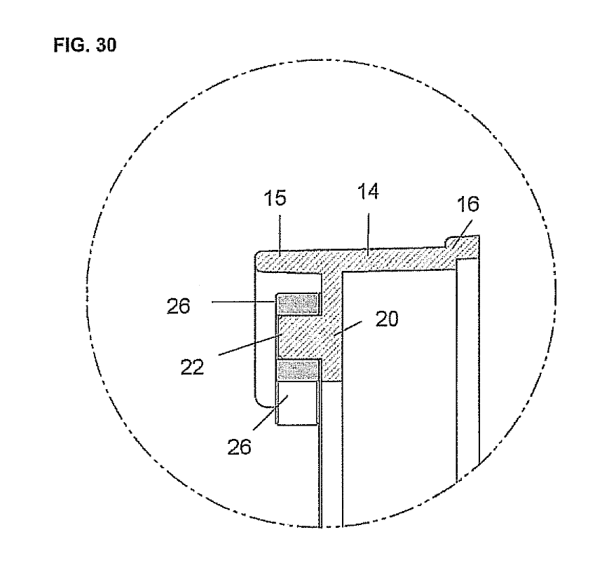

1. A heat sealable member comprising a sheet of heat sealable material to a multiwell plate, wherein the sheet is mounted in a frame, and wherein said frame is sized to fit over a multiwell plate so that the sheet is contacted with at least one of the rims of the top of the plate and the individual wells.

2. The heat sealable member according to claim 1, wherein the frame comprises a rigid polymer.

3. The heat sealable member according to claim 1, wherein the sheet is fixedly mounted to the frame.

4. The heat sealable member according to claim 1, wherein the frame is detachable from the sheet.

5. The heat sealable member according to claim 4, wherein the frame is detachable from the sheet by softening of the sealed material.

6. The heat sealable member according to claim 1, wherein the frame has a skirt portion that extends beneath the sheet.

7. The heat sealable member according to claim 6, wherein the frame has an upstanding wall that extends above the sheet.

8. The heat sealable member according to claim 6, wherein the skirt portion terminates in a lower rim.

9. The heat sealable member according to claim 6, wherein the edge of the upstanding wall is tapered slightly.

10. The heat sealable member according to claim 1, wherein the frame is formed from a material selected from the group consisting of hard plastics, filled polycarbonate, polystyrene, filled polypropylene, metal wire and cardboard.

11. The heat sealable member according to claim 1, wherein the sheet is a multilayer sheet.

12. The heat sealable member according to claim 11, wherein the frame integral with its sheet and formed of the same material as the support layer of a multilayer sheet are formed from the same material.

13. The heat sealable member according to claim 1, wherein sheet is welded or glued to the frame.

14. The heat sealable member according to claim 13, wherein the frame includes a circumextending ledge around an inner side thereof, to the surface of which the sheet is glued or welded.

15. The heat sealable member according to claim 14, wherein the connection between sheet and ledge is loosened upon application of heat.

16. The heat sealable member according to claim 14, wherein the ledge is furnished with a plurality of sharp spikes or pins.

17. The heat sealable member according to claim 16, wherein the sharp spikes or pins are formed integrally with the ledge.

18. The heat sealable member according to claim 14, wherein the ledge is furnished with a plurality of studs, and an apertured second frame part with through openings that correspond at the positions of the studs, and wherein the sheet has apertures at the positions of the studs, so that the second frame part may clip over the studs and the sheet is sandwiched between the two frame portions.

19. The heat sealable member according to claim 14, wherein the ledge is furnished with a plurality of studs, and wherein the sheet has apertures at the positions of the studs, and discrete clips for each stud are holding the sheet in place clip over the studs and the sheet is sandwiched between the two frame portions.

20. The heat sealable member according to claim 18, wherein said member is detachable from their respective stud by a sharp downward push on a main frame portion.

21. A method for sealing a multiwell plate or one or more wells therein comprising:
i.) Placing a heat sealable member according to claim 1 over a top of a multiwell plate
ii.) Applying heat to the sheet or to at least a selected portion thereof to seal the plate as a whole or one or more wells therein.

22. The method of claim 21, further comprising detaching a frame from the multiwell plate sealed with the heat sealable sheet.

23. The method according to claim 22, wherein the heat sealing is performed by a heated platen applied manually, automatically or semi-automatically.

24. A heat sealable member according to claim 1, that is capable of being used with an automatic plate and lid handling apparatus.

25. A heat sealable member according to claim 24 capable of being used in a PCR process.

26. The method according to claim 21, comprising using an automatic plate and lid handling apparatus.

27. The method of claim 21 wherein said method involves a PCR process.
